SEQUENCE LISTING (4 sequences)

SEQ ID NO: 1 - protein, 43 aa, Artificial Sequence
  linker-epitope
SKKKKGARGPESRLLEFYLAMPFATPMEAELARRSLAQDAPPL

SEQ ID NO: 2 - protein, 5 aa, Artificial Sequence
  linker
SKKKK

SEQ ID NO: 3 - protein, 18 aa, Artificial Sequence
  synthetic intermediate
PMEAELARRSLAQDAPPL

SEQ ID NO: 4 - protein, 20 aa, Artificial Sequence
  Synthetic intermediate
ATPMEAELARRSLAQDAPPL

(19) United States
(12) Patent Application Publication
(10) Pub. No.: US 2020/0347108 A1
(43) Pub. Date: Nov. 5, 2020

(54) Peptide Conjugates Incorporating Urea Elements and Their Use as Vaccines

(71) Applicants: SapVax, LLC, Cleveland, OH (US); Auckland UniServices Limited, Auckland (NZ)

(72) Inventors: William Greenlee, Teaneck, NJ (US); Suzanne E. Berezovsky, Cleveland Heights, OH (US); George L. Trainor, Wilmington, DE (US); Margaret A. Brimble, Auckland (NZ); Geoffrey M. Williams, Auckland (NZ)

(21) Appl. No.: 16/864,348

(22) Filed: May 1, 2020

Related U.S. Application Data
(60) Provisional application No. 62/841,893, filed on May 2, 2019.

(51) Int. Cl. C07K 14/47; A61K 39/00

(52) U.S. Cl. CPC C07K 14/4748; A61K 39/001188

(57) ABSTRACT

The present invention provides compounds that may be useful for provoking an immune response against an epitope in a subject in need thereof

### CROSS-REFERENCE TO RELATED APPLICATION

This application claims priority under 35 U.S.C. § 119(e) to U.S. Provisional Patent Application Ser. No. 62/841,893, filed May 2, 2019. The entire content of this application is hereby incorporated by reference herein.

### BACKGROUND OF THE INVENTION

Therapeutic and preventative vaccines are valuable tools that stimulate the immune system for treating diseases, such as cancer, and maintaining public health. There is a continuing need in the art for novel compounds and compositions that can be used to provoke an immune response against a target. The present disclosure addresses this need.

### SUMMARY OF THE INVENTION

In one embodiment, a composition comprising a compound of formula (1):

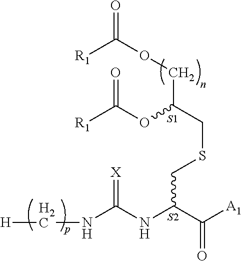

wherein:

each instance of R₁ is independently C5-21 aliphatic or C4-20 heteroaliphatic; A₁ comprises an epitope;

X is O or S;

n is an integer selected from the group consisting of 2-5;

p is an integer selected from the group consisting of 0-15; and

S1 and S2 are each independently R or S; or a pharmaceutically acceptable salt or solvate thereof.

In various embodiments, X is 0.

In various embodiments, wherein p is 12.

In various embodiments, p is 14.

In various embodiments, Si is R.

In various embodiments, Si is S.

In various embodiments, S2 is R.

In various embodiments, S2 is S.

In various embodiments, p is 14; n is 2; and S1 and S2 are R.

In various embodiments, p is 14; n is 3; and S1 and S2 are R.

In various embodiments, p is 14; n is 4; and S1 and S2 are R.

In various embodiments, p is 14; n is 5; and S1 and S2 are R.

In various embodiments, p is 14; n is 2; and S1 and S2 are S.

In various embodiments, p is 14; n is 2; and Si is S and S2 is R.

In various embodiments, Aₗ comprises the amino acids of SEQ ID NO: 2 SKKKK.

In various embodiments, the composition further comprises at least one pharmaceutically acceptable carrier.

In various embodiments, the invention provides a method of provoking or enhancing an immune response in a subject, the method comprising administering to the subject an effective amount of a composition of the invention.

### BRIEF DESCRIPTION OF THE DRAWINGS

The following detailed description of illustrative embodiments of the invention will be better understood when read in conjunction with the appended drawings. For the purpose of illustrating the invention, certain illustrative embodiments are shown in the drawings. It should be understood, however, that the invention is not limited to the precise arrangements and instrumentalities of the embodiments shown in the drawings.

FIG. 1 depicts a schematic overview of the compounds of the invention. Homologation refers to the extension of the alkyl chain at the indicated position. The urea moiety adjacent to cysteine is highlighted. In various embodiments, the chain homologation may be [C2], [C3], [C4], or [C5]; X may be oxygen or sulfur; Y is sulfur; and n is an integer selected from the group consisting of 1-15.

FIG. 2 depicts an overview of the synthesis of a compound of the invention using solid phase peptide synthesis. Step (1) Fmoc-amino acid (5 eq), HATU (4.75 eq), N-Methylmorpholine (NMM) (10 eq), dimethylformamide (DMF), 1 hour. Step (2) 20% piperidine in DMF, 2 washings of 5 minutes. Step (3) R-Fmoc-hPam₂Cys-OH (5 eq), PyBOP (4.75 eq), collidine (10 eq), DMF, room temperature 1-18 hours). Step (4) 20% piperidine in DMF, 10 minutes. Step (5) Tetradecylisocyanate, CH₂Cl₂, 5 hours. Step (6) 2,2′-(Ethylenedioxy)diethanethiol) (DODT) in trifluoroacetic acid (TFA (5% v/v), room temperature, 3 hours (cleavage from resin). The crude peptide thus obtained was then purified.

FIG. 3 is a chromatogram depicting the purified compound of the invention, bearing the antigen SEQ ID NO: 1 SKKKK-GARGPESRLLEFYLAMPFATPMEAELARRSLAQDAPPL (linker-epitope). This was obtained using a Waters XTerra C18 column (5μ; 4.6×150 mm) and a linear gradient of 5-95% B over 30 min, flow rate of 1.0 mL/mi. Buffer A: H₂O containing 0.1% TFA (v/v); Buffer B: acetonitrile containing 0.1% TFA.

FIG. 4 is a low-resolution electrospray ionization mass spectrometry (ESI-MS) of a purified compound of the invention, bearing the antigen SEQ ID NO: 1 SKKKK-GARGPESRLLEFYLAMPFATPMEAELARRSLAQDAPPL (linker-epitope). Calculated mass: 5678.3 (100%) da: 1420.6 [M+4H]⁴⁺, 1136.7 [M+5H]⁵⁺, 947.4 [M+6H]⁶⁺, Found: 5680.5 da: 1421.0 [M+4H]⁴⁺, 1137.2 [M+5H]⁵⁺, 947.8 [M+6H]⁶⁺.

### DETAILED DESCRIPTION OF THE INVENTION

The present invention relates in part to the identification of improved Pam-Cys compounds that may be used as vaccines.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. As used herein, each of the following terms has the meaning associated with it in this section. Generally, the nomenclature used herein and the laboratory procedures in cell culture, molecular genetics, pharmacology and organic chemistry are those well-known and commonly employed in the art.

The methods for SPPS outlined above are well known in the art. See, for example, Atherton and Sheppard, “Solid Phase Peptide Synthesis: A Practical Approach,” New York: IRL Press, 1989; Stewart and Young: “Solid-Phase Peptide Synthesis 2nd Ed.,” Rockford, Ill.: Pierce Chemical Co., 1984; Jones, “The Chemical Synthesis of Peptides,” Oxford: Clarendon Press, 1994; Merrifield, J. Am. Soc. 85:2146-2149 (1963); Marglin, A. and Merrifield, R. B. Annu. Rev. Biochem. 39:841-66 (1970); and Merrifield R. B. JAMA. 210(7):1247-54 (1969); and “Solid Phase Peptide Synthesis—A Practical Approach” (W. C. Chan and P. D. White, eds. Oxford University Press, 2000). Equipment for automated synthesis of peptides or polypeptides is readily commercially available from suppliers such as Perkin Elmer/Applied Biosystems (Foster City, Calif.) and may be operated according to the manufacturer's instructions.

Unless otherwise indicated, conventional techniques of molecular biology, microbiology, cell biology, biochemistry and immunology, which are within the skill of the art may be employed in practicing the methods described herein. Such techniques are explained fully in the literature, such as, Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); The Immunoassay Handbook (David Wild, ed., Stockton Press NY, 1994); Antibodies: A Laboratory Manual (Harlow et al., eds., 1987); and Methods of Immunological Analysis (R. Masseyeff, W. H. Albert, and N. A. Staines, eds., Weinheim: VCH Verlags gesellschaft mbH, 1993).

Standard techniques are used for biochemical and/or biological manipulations. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, N.Y., and Ausubel et al., 2002, Current Protocols in Molecular Biology, John Wiley & Sons, NY), which are provided throughout this document.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “binding” refers to the adherence of molecules to one another, such as, but not limited to, enzymes to substrates, antibodies to antigens, DNA strands to their complementary strands. Binding occurs because the shape and chemical nature of parts of the molecule surfaces are complementary. A common metaphor is the “lock-and-key” used to describe how enzymes fit around their substrate.

An “effective amount” or “therapeutically effective amount” of a compound is that amount of compound sufficient to provide a beneficial effect to the subject to which the compound is administered. An “effective amount” of a delivery vehicle is that amount sufficient to effectively bind or deliver a compound.

As used herein, the term “epitope” refers to the portion of an antigen that is recognized by the immune system.

By “immune response” is meant the actions taken by a host to defend itself from pathogens or abnormalities. The immune response includes innate (natural) immune responses and adaptive (acquired) immune responses. Innate responses are antigen non-specific. Adaptive immune responses are antigen specific.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” or “therapeutically acceptable salt” refers to a salt of the administered compounds prepared from pharmaceutically acceptable non-toxic acids, including inorganic acids or bases, organic acids or bases, solvates, hydrates, or clathrates thereof.

As used herein, the terms “polypeptide,” “protein” and “peptide” are used interchangeably and refer to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

By the term “specifically binds,” as used herein, is meant a molecule, such as an antibody, which recognizes and binds to another molecule or feature, but does not substantially recognize or bind other molecules or features in a sample.

As used herein, the term “alkyl,” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Certain specific examples include (C₁-C₆)alkyl, such as, but not limited to, ethyl, methyl, isopropyl, isobutyl, n-pentyl, n-hexyl and cyclopropylmethyl.

As used herein, the term “cycloalkyl,” by itself or as part of another substituent means, unless otherwise stated, a cyclic chain hydrocarbon having the number of carbon atoms designated (i.e., C₃-C₆ means a cyclic group comprising a ring group consisting of three to six carbon atoms) and includes straight, branched chain or cyclic substituent groups. Examples include cyclopropyl, cyclobutyl, cyclopentyl, cyclohexyl, cycloheptyl, and cyclooctyl. Certain specific examples include (C₃-C₆)cycloalkyl, such as, but not limited to, cyclopropyl, cyclobutyl, cyclopentyl and cyclohexyl.

As used herein, the term “substituted alkyl” or “substituted cycloalkyl” means alkyl or cycloalkyl, as defined above, substituted by one, two or three substituents selected from the group consisting of halogen, —OH, alkoxy, tetrahydro-2-H-pyranyl, —NH₂, —N(CH₃)₂, (1-methyl-imidazol-2-yl), pyridin-2-yl, pyridin-3-yl, pyridin-4-yl, —C(═O)OH, trifluoromethyl, —C≡N, —C(═O)O(C₁-C₄)alkyl, —C(═O)NH₂, —C(═O)NH(C₁-C₄)alkyl, —C(═O)N((C₁-C₄)alkyl)₂, —SO₂NH₂, —C(═NH)NH₂, and —NO₂, advantageously containing one or two substituents selected from halogen, —OH, alkoxy, —NH₂, trifluoromethyl, —N(CH₃)₂, and —C(═O)OH, more advantageously selected from halogen, alkoxy and —OH. Examples of substituted alkyls include, but are not limited to, 2,2-difluoropropyl, 2-carboxycyclopentyl and 3-chloropropyl.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. In certain embodiments, alkoxy includes (C₁-C₃)alkoxy, such as, but not limited to, ethoxy and methoxy.

As used herein, the term “halo” or “halogen” alone or as part of another substituent means, unless otherwise stated, a fluorine, chlorine, bromine, or iodine atom, advantageously, fluorine, chlorine, or bromine, more advantageously, fluorine or chlorine.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “aromatic” refers to a carbocycle or heterocycle with one or more polyunsaturated rings and having aromatic character, i.e. having (4n+2) delocalized π (pi) electrons, where n is an integer. As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. In certain embodiments, aryl includes phenyl and naphthyl, in particular, phenyl.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (such as, but not limited to, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “substituted” means that an atom or group of atoms has replaced hydrogen as the substituent attached to another group.

For aryl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet another embodiments, the substituents vary in number between one and two. In yet another embodiments, the substituents are independently selected from the group consisting of C₁₋₆ alkyl, —OH, C₁₋₆ alkoxy, halo, amino, acetamido and nitro. As used herein, where a substituent is an alkyl or alkoxy group, the carbon chain may be branched, straight or cyclic, in particular, straight.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds and Compositions

In one aspect, the invention provides a composition comprising a compound of formula (1):

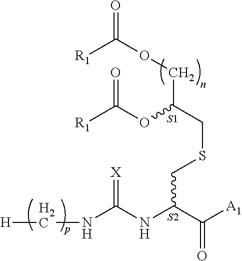

wherein:

each instance of R₁ is independently C5-21 aliphatic or C4-20 heteroaliphatic;

A₁ comprises an epitope;

X is O or S;

n is an integer selected from the group consisting of 2-5;

p is an integer selected from the group consisting of 0-15; and

S1 and S2 each independently indicate that the adjacent carbon atom may have either the R or S absolute configuration;

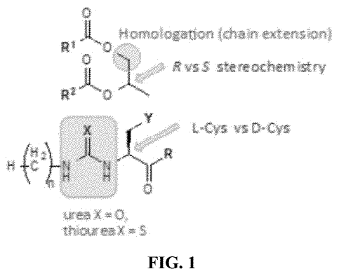

or a pharmaceutically acceptable salt or solvate thereof. In various embodiments, X is oxygen. In various embodiments, p is 12. In various embodiments, p is 14. FIG. 1 depicts a schematic view of the structure of the compounds of the invention marking certain features of these compounds.

S1 and S2 refer to the adjacent stereogenic carbons (wavy bond). In various embodiments, the stereogenic carbon at S1 is the R or S stereoisomer and the stereochemistry at S2 is independently the R or S stereoisomer. The cysteine residue fits tightly in the TLR binding pocket, therefore a change in stereochemistry at this position (from L-Cys to D-Cys) could alter activity significantly. In various embodiments, this may be used as a method of enhancing or mitigating activity, or improving the pharmacokinetic profile of the compound.

In various embodiments, p is 14; n is 2; and S1 and S2 are R. In various embodiments, p is 14; n is 3; and S1 and S2 are R. In various embodiments, p is 14; n is 4; and Si and S2 are R. In various embodiments, p is 14; n is 5; and S1 and S2 are R. In various embodiments, p is 14; n is 2; and S1 and S2 are S. In various embodiments, S1 is S and S2 is R.

In various embodiments, A₁ comprises an epitope. The epitope may be any epitope known in the art. In various embodiments, the epitope is a peptide epitope. In various embodiments, A₁ may comprise a peptide linker between cysteine and the epitope. In various embodiments, comprises the amino acids SEQ ID NO: 2 SKKKK as a linker. Various exemplary antigens from which epitopes may be derived, each of which may be incorporated into compounds of the invention are described below.

In various embodiments, the composition further comprises at least one pharmaceutically acceptable carrier. In various embodiments, the invention provides a method of provoking an immune response in a subject, the method comprising administering to the subject an effective amount of a composition according to the invention. The epitope at Aₗ may correspond to an epitope on the antigen against which the immune response is to be provoked. Details of administration and dose are discussed in further detail below.

### Antigens

It will be appreciated that a great many antigens, for example tumour antigens or antigens from various pathogenic organisms, or allergens, have been characterised and are suitable for use in the present invention. All antigens, whether or not presently characterized, that are capable of eliciting an immune response are contemplated.

Accordingly, depending on the choice of antigen the conjugates of the present invention find application in a wide range of immunotherapies, including but not limited to the treatment and prevention of infectious disease, the treatment and prevention of cancer, the treatment and prevention of allergies, and the treatment of viral re-activation during or following immunosuppression, for example in patients who have had bone marrow transplants or haematopoietic stem cell transplants.

Also contemplated are antigens comprising one or more amino acid substitutions, such as one or more conservative amino acid substitutions.

Those of skill in the art will appreciate that the conjugates of the present invention are in certain embodiments particularly suited for stimulating T-cell responses, for example in the treatment of neoplastic diseases, including cancer. Conjugates of the present invention comprising one or more tumour antigens are specifically contemplated. It will be appreciated that tumour antigens contemplated for use in the preparation of peptide conjugates of the invention will generally comprise one or more epitope containing peptides. In certain embodiments of the invention, including for example pharmaceutical compositions of the invention, one or more additional tumour antigens may be present, wherein the one or more tumour antigens does not comprise peptide. Tumour antigens are typically classified as either unique antigens, or shared antigens, with the latter group including differentiation antigens, cancer-specific antigens, and over-expressed antigens. Examples of each class of antigens are amenable to use in the present invention. Representative tumour antigens for use in the treatment, for example immunotherapeutic treatment, or vaccination against neoplastic diseases including cancer, are discussed below. Compounds, vaccines and compositions comprising one or more antigens prepared using those methods of immunisation are specifically contemplated.

In certain embodiments, the tumour antigen is peptide-containing tumour antigen, such as a polypeptide tumour antigen or glycoprotein tumour antigens. In certain embodiments, the tumour antigen is a saccharide-containing tumour antigen, such as a glycolipid tumour antigen or a ganglioside tumour antigen. In certain embodiments, the tumour antigen is a polynucleotide-containing tumour antigen that expresses a polypeptide-containing tumour antigen, for instance, an RNA vector construct or a DNA vector construct, such as plasmid DNA.

Tumour antigens appropriate for use in the present invention encompass a wide variety of molecules, such as (a) peptide-containing tumour antigens, including peptide epitopes (which can range, for example, from 8-20 amino acids in length, although lengths outside this range are also common), lipopolypeptides and glycoproteins, (b) saccharide-containing tumour antigens, including poly-saccharides, mucins, gangliosides, glycolipids and glycoproteins, including and (c) polynucleotides that express antigenic polypeptides. Again, those skilled in the art will recognise that a tumour antigen present in a conjugate or composition of the present invention will typically comprise a peptide. However, embodiments of the invention where one or more conjugates comprises a tumour antigen that does not itself comprise a peptide, but for example is a non-peptide chemical structure bound to the amino acid-comprising or peptide-containing conjugation partner, are contemplated. Similarly, compositions of the invention in which one or more tumour antigens that does not itself comprise peptide is present are contemplated. In certain embodiments, the tumour antigens are, for example, (a) full length molecules associated with cancer cells, (b) homologues and modified forms of the same, including molecules with deleted, added and/or substituted portions, and (c) fragments of the same, provided said fragments remain antigenic or immunogenic. In certain embodiments, the tumour antigens are provided in recombinant form. In certain embodiments, the tumour antigens include, for example, class I-restricted antigens recognized by CD8+ lymphocytes or class II-restricted antigens recognized by CD4+ lymphocytes. In certain embodiments, tumor antigens include synthetic peptides comprising class I-restricted antigens recognized by CD8+ lymphocytes or class II-restricted antigens recognized by CD4+ lymphocytes.

Shared tumour antigens are generally considered to be native, unmutated sequences that are expressed by tumours due to epigenetic changes that allow de-repression of developmentally-repressed genes. Accordingly, shared antigens are typically considered preferable to over-expressed or differentiation-associated antigens because there is no or low expression in most normal tissues. Also, the same antigens can be targeted in a number of cancer patients. For example, the cancer-testis antigen NY-ESO-1 is present in the majority of patients with many tumours, and a sizeable minority of patients with other tumours. In another example, breast differentiation tumour antigens NYBR-1 and NYBR-1.1 are found in a proportion of breast cancer sufferers. Shared tumour antigens thus represent an attractive target for development.

The use of shared tumour antigens, such cancer-testis antigens including NY-ESO-1, CTSP-1, CTSP-2, CTSP-3, CTSP-4, SSX2, and SCP1, and breast cancer antigens NYBR-1 and NYBR-1.1, in conjugates of the present invention is specifically contemplated herein.

Similarly, the prostate vaccine Sipuleucel-T (APC8015, Provenge™), which comprises the antigen prostatic acid phosphatase (PAP), is present in 95% of prostate cancer cells. At least in part due to this potential for efficacy in a significant proportion of prostate cancer sufferers, Sipuleucel-T was approved by the FDA in 2010 for use in the treatment of asymptomatic, hormone-refractory prostate cancer. The use of PAP antigen in conjugates of the present invention is specifically contemplated in the present invention.

Unique antigens are considered to be those antigens that are unique to an individual or are shared by a small proportion of cancer patients, and typically result from mutations leading to unique protein sequences. Representative examples of unique tumour antigens include mutated Ras antigens, and mutated p53 antigens. As will be appreciated by those skilled in the art having read this specification, the methods of the present invention enable the ready preparation of conjugates comprising one or more unique tumour antigens, for example to elicit specific T-cell responses to one or more unique tumour antigens, for example in the preparation of patient-specific therapies.

Accordingly, representative tumour antigens include, but are not limited to, (a) antigens such as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumours), (b) mutated antigens, for example, p53 (associated with various solid tumours, for example, colorectal, lung, head and neck cancer), p21/Ras (associated with, for example, melanoma, pancreatic cancer and colorectal cancer), CDK4 (associated with, for example, melanoma), MUM1 (associated with, for example, melanoma), caspase-8 (associated with, for example, head and neck cancer), CIA 0205 (associated with, for example, bladder cancer), HLA-A2-R1701, beta catenin (associated with, for example, melanoma), TCR (associated with, for example, T-cell non-Hodgkins lymphoma), BCR-abl (associated with, for example, chronic myelogenous leukemia), triosephosphate isomerase, MA 0205, CDC-27, and LDLR-FUT, (c) over-expressed antigens, for example, Galectin 4 (associated with, for example, colorectal cancer), Galectin 9 (associated with, for example, Hodgkin's disease), proteinase 3 (associated with, for example, chronic myelogenous leukemia), Wilm's tumour antigen-1 (WT 1, associated with, for example, various leukemias), carbonic anhydrase (associated with, for example, renal cancer), aldolase A (associated with, for example, lung cancer), PRAME (associated with, for example, melanoma), HER-2/neu (associated with, for example, breast, colon, lung and ovarian cancer), alpha-fetoprotein (associated with, for example, hepatoma), KSA (associated with, for example, colorectal cancer), gastrin (associated with, for example, pancreatic and gastric cancer), telomerase catalytic protein, MUC-1 (associated with, for example, breast and ovarian cancer), G-250 (associated with, for example, renal cell carcinoma), p53 (associated with, for example, breast, colon cancer), and carcinoembryonic antigen (associated with, for example, breast cancer, lung cancer, and cancers of the gastrointestinal tract such as colorectal cancer), (d) shared antigens, for example, melanoma-melanocyte differentiation antigens such as MART-1/Melan A, gp100, MC1R, melanocyte-stimulating hormone receptor, tyrosinase, tyrosinase related protein-1/TRP1 and tyrosinase related protein-2/TRP2 (associated with, for example, melanoma), (e) prostate associated antigens such as PAP, prostatic serum antigen (PSA), PSMA, PSH-P1, PSM-P1, PSM-P2, associated with for example, prostate cancer, (f) immunoglobulin idiotypes (associated with myeloma and B cell lymphomas, for example), and (g) other tumour antigens, such as polypeptide- and saccharide-containing antigens including (i) glycoproteins such as sialyl Tn and sialyl Le.sup.x (associated with, for example, breast and colorectal cancer) as well as various mucins; glycoproteins are coupled to a carrier protein (for example, MUC-1 are coupled to KLH); (ii) lipopolypeptides (for example, MUC-1 linked to a lipid moiety); (iii) polysaccharides (for example, Globo H synthetic hexasaccharide), which are coupled to a carrier proteins (for example, to KLH), (iv) gangliosides such as GM2, GM12, GD2, GD3 (associated with, for example, brain, lung cancer, melanoma), which also are coupled to carrier proteins (for example, KLH).

Other representative tumour antigens amenable to use in the present invention include TAG-72, (See, e.g., U.S. Pat. No. 5,892,020; human carcinoma antigen (See, e.g., U.S. Pat. No. 5,808,005); TP1 and TP3 antigens from osteocarcinoma cells (See, e.g., U.S. Pat. No. 5,855,866); Thomsen-Friedenreich (TF) antigen from adenocarcinoma cells (See, e.g., U.S. Pat. No. 5,110,911); KC-4 antigen from human prostrate adenocarcinoma (See, e.g., U.S. Pat. No.

4,743,543); a human colorectal cancer antigen (See, e.g., U.S. Pat. No. 4,921,789); CA125 antigen from cystadenocarcinoma (See, e.g., U.S. Pat. No. 4,921,790); DF3 antigen from human breast carcinoma (See, e.g., U.S. Pat. Nos. 4,963,484 and 5,053,489); a human breast tumour antigen (See, e.g., U.S. Pat. No. 4,939,240); p97 antigen of human melanoma (See, e.g., U.S. Pat. No. 4,918,164); carcinoma or orosomucoid-related antigen (CORA) (See, e.g., U.S. Pat. No. 4,914,021); T and Tn haptens in glycoproteins of human breast carcinoma, MSA breast carcinoma glycoprotein; MFGM breast carcinoma antigen; DU-PAN-2 pancreatic carcinoma antigen; CA125 ovarian carcinoma antigen; YH206 lung carcinoma antigen, Alphafetoprotein (AFP) hepatocellular carcinoma antigen; Carcinoembryonic antigen (CEA) bowel cancer antigen; Epithelial tumour antigen (ETA) breast cancer antigen; Tyrosinase; the raf oncogene product; gp75; gp100; EBV-LMP 1 & 2; EBV-EBNA 1, 2 & 3C; HPV-E4, 6, 7; CO17-1A; GA733; gp72; p53; proteinase 3; telomerase; and melanoma gangliosides. These and other tumour antigens, whether or not presently characterized, are contemplated for use in the present invention.

In certain embodiments, the tumour antigens are derived from mutated or altered cellular components. Representative examples of altered cellular components include, but are not limited to ras, p53, Rb, altered protein encoded by the Wilms' tumour gene, ubiquitin, mucin, protein encoded by the DCC, APC, and MCC genes, as well as receptors or receptor-like structures such as neu, thyroid hormone receptor, platelet derived growth factor (PDGF) receptor, insulin receptor, epidermal growth factor (EGF) receptor, and the colony stimulating factor (CSF) receptor.

Polynucleotide-containing antigens used in the present invention include polynucleotides that encode polypeptide tumour antigens such as those listed above. In certain embodiments, the polynucleotide-containing antigens include, but are not limited to, DNA or RNA vector constructs, such as plasmid vectors (e.g., pCMV), which are capable of expressing polypeptide tumour antigens in vivo.

The present invention also contemplates the preparation of conjugates comprising viral antigens that are capable of stimulating T-cell to elicit effective anti-viral immunity in patients who are or have been immunosuppressed, for example elderly patients or patients who have had bone marrow transplants, haematopoietic stem cell transplants, or are otherwise undergoing immunosuppression.

Similarly, antigens derived from viruses associated with increased incidence of cancer, or that are reported to be cancer-causing, such as human papillomavirus, hepatitis A virus, and hepatitis B virus, are contemplated for use in the present invention.

For example, in certain embodiments, the tumour antigens include, but are not limited to, p15, Hom/Me1-40, H-Ras, E2A-PRL, H4-RET, IGH-IGK, MYL-RAR, Epstein Barr virus antigens, human papillomavirus (HPV) antigens, including E6 and E7, hepatitis B and C virus antigens, human T-cell lymphotropic virus antigens, TSP-180, p185erbB2, p180erbB-3, c-met, mn-23H1, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29\BCAA), CA 195, CA 242, CA-50, CAM43, CD68\KP1, CO-029, FGF-5, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, TPS, and the like.

In certain embodiments, the tumour antigens include viral proteins implicated in oncogenesis, such as antigens from Epstein Barr virus, human papillomavirus (HPV), including E6 and E7, and hepatitis B and C, and human T-cell lymphotropic virus.

It will be appreciated that such viral proteins, as well as various other viral proteins can also be targets for T cell activity in, for example, treatment against viral disease. In fact, the present invention may be useful in any infection where T cell activity is known to play a role in immunity (effectively all virus infections and many bacterial infections as well, such as tuberculosis). The infectious diseases described herein are provided by way of example only and are in no way intended to limit the scope of the invention. It will be appreciated that the present invention may be useful in the treatment of various other diseases and conditions.

Representative antigens for use in vaccination against pathogenic organisms are discussed below. Compounds, vaccines and compositions comprising one or more antigens prepared using those methods of immunisation are specifically contemplated.

### Tuberculosis Antigens

It will be appreciated that a great many M tuberculosis antigens have been characterised and are suitable for use in the present invention. All M tuberculosis antigens, whether or not presently characterized, that are capable of eliciting an immune response are contemplated.

Exemplary M tuberculosis antigens suitable for use include early secretary antigen target (ESAT)-6, Ag85A, Ag85B (MPT59), Ag85B, Ag85C, MPT32, MPT51, MPT59, MPT63, MPT64, MPT83, MPB5, MPB59, MPB64, MTC28, Mtb2, Mtb8.4, Mtb9.9, Mtb32A, Mtb39, Mtb41, TB10.4, TB10C, TB11B, TB12.5, TB13A, TB14, TB15, TB15A, TB16, TB16A, TB17, TB18, TB21, TB20.6, TB24, TB27B, TB32, TB32A, TB33, TB38, TB40.8, TB51, TB54, TB64, CFP6, CFP7, CFP7A, CFP7B, CFP8A, CFP8B, CFP9, CFP10, CFP11, CFP16, CFP17, CFP19, CFP19A, CFP19B, CFP20, CFP21, CFP22, CFP22A, CFP23, CFP23A, CFP23B, CFP25, CFP25A, CFP27, CFP28, CFP28B, CFP29, CFP30A, CFP30B, CFP50, CWP32, hspX (alpha-crystalline), APA, Tuberculin purified protein derivative (PPD), ST-CF, PPE68, LppX, PstS-1, PstS-2, PstS-3, HBHA, GroEL, GroEL2, GrpES, LHP, 19kDa lipoprotein, 71kDa, RD1-ORF2, RD1-ORF3, RD1-ORF4, RD1-ORFS, RD1-ORFS, RD1-ORF9A, RD1-ORF9B, Rv1984c, Rv0577, Rv1827, BfrB, Tpx. Rv1352, Rv1810, PpiA, Cut2, FbpB, FbpA, FbpC, DnaK, FecB, Ssb, Rp1L, FixA, FixB, AhpC2, Rv2626c, Rv1211, Mdh, Rv1626, Adk, ClpP, SucD (Belisle et al, 2005; US 7,037,510; US 2004/0057963; US 2008/0199493; US 2008/0267990), or at least one antigenic portion or T-cell epitope of any of the above mentioned antigens.

### Hepatitis Antigens

A number of hepatitis antigens have been characterised and are suitable for use in the present invention. Exemplary hepatitis C antigens include C—p22, E1—gp35, E2—gp70, NS1—p7, NS2—p23, NS3—p′70, NS4A—p8, NS4B—p27, NS5A—p56/58, and NS5B—p68, and together with one or more antigenic portions or epitopes derived therefrom are each (whether alone or in combination) suitable for application in the present invention. All hepatitis antigens, whether or not presently characterized, that are capable of eliciting an immune response are contemplated.

### Influenza Antigens

Many influenza antigens have been characterised and are suitable for use in the present invention. Exemplary influenza antigens suitable for use in the present invention include PB, PB2, PA, any of the hemagglutinin (HA) or neuramimidase (NA) proteins, NP, M, and NS, and together with one or more antigenic portions or epitopes derived therefrom are each (whether alone or in combination) suitable for application in the present invention. All influenza antigens, whether or not presently characterized, that are capable of eliciting an immune response are contemplated.

### Anthrax Antigens

A number of B. anthracis antigens have been identified as potential candidates for vaccine development and are useful in the present invention. For example, PA83 is one such antigen for vaccine development. Currently, only one FDA licensed vaccine for anthrax is available called “Anthrax Vaccine Adsorbed” (AVA) or BioThrax®. This vaccine is derived from the cell-free supernatant of a non-encapsulated strain of B. anthracis adsorbed to aluminum adjuvant. PA is the primary immunogen in AVA. Other exemplary anthrax antigens suitable for use in the present invention include Protective antigen (PA or PA63), LF and EF (proteins), poly-gamma-(D-glutamate) capsule, spore antigen (endospore specific components), Bc1A (exosporium specific protein), BxpB (spore-associated protein), and secreted proteins. All anthrax antigens together with one or more antigenic portions or epitopes derived therefrom, whether or not presently characterized, that are capable of eliciting an immune response are contemplated.

### Tularemia Antigens

A number of F. tularensis antigens have been identified as potential candidates for vaccine development and are useful in the present invention. For example, AcpA and Ig1C are antigens suitable for vaccine development. Other exemplary Tularemia antigens suitable for use in the present invention include O-antigen, CPS, outer membrane proteins (e.g. FopA), lipoproteins (e.g. Tu14), secreted proteins and lipopolysaccharide. All tularemia antigens together with one or more antigenic portions or epitopes derived therefrom, whether or not presently characterized, that are capable of eliciting an immune response are contemplated.

### Brucellosis Antigens

A number of B. abortusis antigens have been identified as potential candidates for vaccine development and are useful in the present invention. For example, Omp16 is one such antigen for vaccine development. Other exemplary Brucellosis antigens suitable for use in the present invention include O-antigen, lipopolysaccharide, outer membrane proteins (e.g. Omp16), secreted proteins, ribosomal proteins (e.g. L7 and L12), bacterioferritin, p39 (a putative periplasmic binding protein), groEL(heat-shock protein), lumazine synthase, BCSP31 surface protein, PAL16.5 OM lipoprotein, catalase, 26 kDa periplasmic protein, 31 kDa Omp31, 28 kDa Omp, 25 kDa Omp, and 10 kDA Om lipoprotein. All brucellosis antigens together with one or more antigenic portions or epitopes derived therefrom, whether or not presently characterized, that are capable of eliciting an immune response are contemplated.

### Meningitis Antigens

A number of N. meningitidis antigens have been identified as potential candidates for vaccine development and are useful in the present invention. For example, Cys6, PorA, PorB, FetA, and ZnuD are antigens suitable for vaccine development. Other exemplary Meningitis antigens suitable for use in the present invention include 0-antigen, factor H binding protein (fHbp), TbpB, NspA, NadA, outer membrane proteins, group B CPS, secreted proteins and lipopolysaccharide. All menigitis antigens together with one or more antigenic portions or epitopes derived therefrom, whether or not presently characterized, that are capable of eliciting an immune response are contemplated.

### Dengue Antigens

A number of Flavivirus antigens have been identified as potential candidates for vaccine development to treat dengue fever and are useful in the present invention. For example, dengue virus envelope proteins E1-E4 and the membrane proteins M1-M4 are antigens suitable for vaccine development. Other exemplary dengue antigens suitable for use in the present invention include C, preM, 1, 2A, 2B, 3, 4A, 4B and 5. All dengue antigens together with one or more antigenic portions or epitopes derived therefrom, whether or not presently characterized, that are capable of eliciting an immune response are contemplated.

### Ebola Antigens

A number of ebola virus antigens have been identified as potential candidates for vaccine development to treat ebola infection and are useful in the present invention. For example, Filoviridae Zaire ebolavirus and Sudan ebolavirus virion spike glycoprotein precursor antigens ZEBOV-GP, and SEBOV-GP, respectively, are suitable for vaccine development. Other exemplary ebola antigens suitable for use in the present invention include NP, vp35, vp40, GP, vp30, vp24 and L. All ebola antigens together with one or more antigenic portions or epitopes derived therefrom, whether or not presently characterized, that are capable of eliciting an immune response are contemplated.

### West Nile Antigens

A number of West Nile virus antigens have been identified as potential candidates for vaccine development to treat infection and are useful in the present invention. For example, Flavivirus envelope antigen (E) from West Nile virus (WNV) is a non-toxic protein expressed on the surface of WNV virions (WNVE) and are suitable for vaccine development. Other exemplary WNV antigens suitable for use in the present invention include Cp, Prm, NS1, NS2A, NS2B, NS3, NS4A, NS4B and NS5.

All West Nile antigens together with one or more antigenic portions or epitopes derived therefrom, whether or not presently characterized, that are capable of eliciting an immune response are contemplated.

The above-listed or referenced antigens are exemplary, not limiting, of the present invention.

### Administration/Dosing

In clinical settings, delivery systems for the compositions described herein can be introduced into a subject by any of a number of methods, each of which is familiar in the art. For instance, a pharmaceutical formulation of the composition can be administered locally, e.g. by inhalation or subcutaneous injection, or systemically, e.g. by intravenous injection.

The regimen of administration may affect what constitutes an effective amount. The therapeutic formulations may be administered to the subject either prior to or after the manifestation of symptoms associated with the disease or condition. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the composition of the present invention to a subject, preferably a mammal, more preferably a human, may be carried out using known procedures, at dosages and for periods of time effective to treat a disease or condition in the subject. An effective amount of the composition necessary to achieve a therapeutic effect may vary according to factors such as the time of administration; the duration of administration; other drugs, compounds or materials used in combination with the composition; the state of the disease or disorder; age, sex, weight, condition, general health and prior medical history of the subject being treated; and like factors well-known in the medical arts. Dosage regimens may be adjusted to provide the optimum therapeutic response. For example, several divided doses may be administered daily or the dose may be proportionally reduced as indicated by the exigencies of the therapeutic situation. One of ordinary skill in the art would be able to study the relevant factors and make the determination regarding the effective amount of the composition without undue experimentation.

Actual dosage levels of pharmaceutical formulations of this invention may be varied so as to obtain an amount of the composition that are effective to achieve the desired therapeutic response for a particular subject, composition, and mode of administration, without being toxic to the subject.

### EXPERIMENTAL EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

### Example 1: Synthesis and Purification

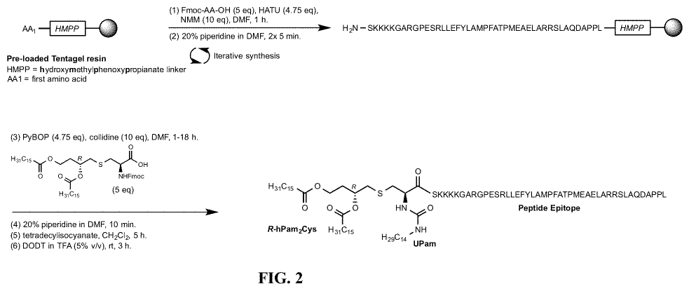

Synthesis of the urea motif was achieved by a straightforward reaction outlined in FIG. 2. First, conventional solid-supported peptide synthesis was used to generate the peptide epitope (in this case a long ESO 79-116 sequence) and the (R)-Fmoc-hPam₂Cys [C2] building block then incorporated to give, after removal of the Fmoc group, the on-resin (R)-hPam2Cys [C2] construct. This material was then treated with tetradecylisocyanate to install the urea group and this product was then cleaved from the resin. After cleavage from resin the material was purified by RP-HPLC. A solution of the crude UPam(R)-hPam₂Cys [C2] peptide of sequence SEQ ID NO: 1 SKKKK- GARGPESRLLEFYLAMPFATPMEAELARRSLAQDAPPL (linker-epitope(ESO[79-116]) was prepared in 1:1 (v/v) water:acetonitrile (containing 0.1%TFA) to a concentration of 10 mg/mL. 0.5 mL aliquots of this solution were purified by high-pressure liquid chromatography using a Phenomenex Gemini C18 5 m 110A, 10×250 mm column with eluent A being water/0.1% TFA (v/v), eluent B being acetonitrile/0.1% TFA (v/v) and generating the following gradient: 0-1 min, 50% B; 1-2 min, 82% B; 2-10 min 95% B; 10-10.5 min 50% B at a flow of 4 mL/min. The peak eluting at approximately 7-8 minutes, corresponding to the desired material, was collected. The process was repeated as necessary and the fractions containing product were pooled and lyophilised.

### Materials

9-Fluorenylmethoxycarbonyl (Fmoc) protected L-R-amino acids, (2-(6-Chloro-1H-benzotriazole-1-yl)-1,1,3,3-tetramethylaminium hexafluorophosphate) (HCTU), benzotriazol-1-yl-oxytripyrrolidinophosphonium hexafluorophosphate (PyBOP) and N-[(Dimethylamino)-1H-1,2,3-triazolo-[4,5-b]pyridin-1-ylmethylene]-N-methylmethanaminium hexafluorophosphate N-oxide (HATU) were purchased from GL Biochem (Shanghai, China). Dimethylformamide (DMF, Scharlau), was purchased from Global Scientific. Piperidine, collidine and diisopropylamine were purchased from Sigma-Aldrich.
Acetonitrile, Methanol, Diethyl ether and Dichloromethane were obtained from Merck (New Zealand), 3,6-Dioxa-1,8-octanedithiol (DODT), triisopropylsilane (TIPS), diisopropylethylamine (DIPEA), and trifluoromethanesulfonic acid (TFMSA) and all other reagents were purchased from Sigma-Aldrich.
Preloaded Fmoc-Leu-wang-Tentagel was obtained from Rapp Polymere (Tuebingen).

### Peptide Synthesis

Part 1: Synthesis of first portion ESO[99-116]
(SEQ ID NO: 3 PMEAELARRSLAQDAPPL-Wang-polystyrene)

This portion of the synthesis was carried out on a 0.2 mmol scale using a Tribute synthesiser (Gyros Protein Technologies, Tucson, Ariz.). A general description of the coupling procedure for a single amino acid follows:
(i) Single coupling The Fmoc-protected Amino Acid (1.0 mmol, 5 eq. relative to resin) and HCTU (397 mg, 0.98 mmol) were combined and dissolved in DMF (approx. 2.0 mL). The mixture was then activated by the addition of 4-methylmorpholine (1.0 mL of a 2 M solution in DMF, 2.0 mmol) and this solution after 2 minutes was transferred to the pre-swollen (DMF) resin. In the case of Methionine HATU was used in place of HCTU as coupling agent in the same proportions.
The resin was gently agitated for 60 minutes, a solution of acetic anhydride (1 mL of a 20% v/v solution in DMF) added and agitation continued for a further one minute before the resin was drained and washed (DMF).
The Fmoc protecting group was then removed by washing the resin with 20% (v/v) piperidine in DMF (2×(4 mL×10 min))
(ii) Double/Triple coupling: (step 1) The Fmoc-protected Amino Acid (1 mmol, 5 eq. relative to resin) and HCTU (397 mg, 0.98 mmol) were combined and dissolved in DMF (˜2 mL). The mixture was then activated by the addition of 4-methylmorpholine (1.0 mL of a 2 M solution in DMF, 2.0 mmol) and this solution after 1 minute was transferred to the pre-swollen (DMF) resin.
The resin was gently agitated for 60 minutes, drained and washed once with DMF. For a triple coupling this step 1 is repeated once again before proceeding to step 2; for a double coupling step 2 is followed directly.
(step 2) This coupling procedure was repeated using the same amino acid, allowing the coupling to proceed for the same length of time (60 min) whereupon a solution of acetic anhydride (1 mL of a 20% v/v solution in DMF) added and agitation continued for a further one minute before the resin was drained and washed (DMF).
On completion The Fmoc protecting group was then removed by washing the resin with 20% (v/v) piperidine in DMF (2 ×(4 mL×10 min))

Part 2: Introduction of [97]A1a[98]Thr using
the corresponding pseudoproline SEQ ID NO: 4
(ATPMEAELARRSLAQDAPPL-Wang-polystyrene)

A solution of Fmoc-Ala-Thr(ψMe,Me-pro)-OH (181 mg, 0.4 mmol) and HATU (152 mg, 0.4 mmol) in DMF (1.8 mL) was activated by addition of 4-methylmorpholine (88 μL, 0.8 mmol) and the resulting solution transferred to the pre-swollen (DMF) resin (0.2 mmol). After gently agitating for 120 minutes the resin was drained and washed (DMF).
A test cleavage showed some unreacted material so the process was repeated. Acetic anhydride (20% v/v in DMF, 1 mL) was added at the end of the repeat coupling The Fmoc protecting group was then removed by washing the resin with 20% (v/v) piperidine in DMF (2×(4 mL×10 min))

Part 3: Addition of residues SEQ ID NO: 2
SKKKK-79-96 (SEQ ID NO: 1 SKKKK-GARG...
DAPPL-Wang-PS)

The synthesis was continued using the Tribute synthesiser according to the procedure described in Part 1 above.

Part 4: Introduction of the adjuvant
component (R)-hPam₂Cys[C2]
(R)-hPam₂Cys[C2](NH2)-SEQ ID NO: 1
SKKKK-GARG....DAPPL-Wang-PS)

A sample of resin (0.12 mmol) was pre-swollen in DMF and drained. (R)-Fmoc-hPam₂Cys[C2]-OH (109 mg, 0.12 mmol) and PyBOP (63 mg, 0.12 mmol) were dissolved in 0.6 mL of DMF and activated by the addition of collidine (32 μL, 0.24 mmol). After mixing for 1 minute the solution was transferred to the pre-swollen resin, which was agitated for 20 hours before being drained and washed.
The Fmoc protecting group was then removed by washing the resin with 20% (v/v) piperidine in DMF (10 mL×3 min then 10 mL×7 min)

Part 5: Addition of the Urea (Upam)
(R)-UPam₂Cys[C2 SEQ ID NO: 1 SKKKK-GARG....
DAPPL-Wang-PS)

A sample of resin (0.06 mmol) was suspended in CH₂Cl₂ (1 mL) and NMP (1 mL) added followed by Tetradecylisocyanate (135 uL, 0.5 mmol). The mixture was agitated gently for 18 h., the resin then drained and washed.

### Cleavage of Peptide from Resin

### (R)-UPam₂Cys[C2]-SEQ ID NO: 1 SKKKK-GARGPESRLLEFYLAMPFATPMEAELARRSLAQDAPPL

The resin (0.06 mmol) was transferred to a sintered glass cleavage funnel equipped with drainage stopcock and screw-cap, washed with dichloromethane and allowed to air-dry.
3 mL cleavage medium comprised of (by volume) 5% water and 95% trifluoroacetic acid (TFA) was prepared and added to the resin. The cleavage vessel was sealed and agitated at room temperature for 150 minutes. The supernatant was then drained into chilled ether (25 mL) and the resin washed with a further 1.5 mL of TFA, which was also drained into the ethereal mixture. The precipitated material was pelleted by centrifugation and the ether supernatant discarded. The pellet was washed once with ether (20 mL) and allowed to dry. The crude peptide was then dissolved in about 15 mL 2:1 MeCN/water (v/v) containing 0.1% TFA and the solution heated at 70° C. for 30 minutes. On cooling the solution was frozen in liquid nitrogen and freeze-dried to afford approximately 142 mg crude peptide.

### Purification of the Peptide

Sample preparation: 10mg of crude, freeze-dried peptide was suspended in 500 uL MeCN/0.1% TFA and water/0.1% TFA (500 uL) was added to induce dissolution, giving a 10 mg/mL solution. The sample was centrifuged and 500 uL aliquots of the supernatant loaded on to the semi-prep column and purified using the given gradient. The peak eluting at approximately 7-8 minutes, corresponding to the desired material, was collected. The process was repeated as necessary and the fractions containing product were pooled and lyophilized. After processing 120 mg of crude material, 38 mg of purified peptide was obtained.

### HPLC Elution Conditions

### Column: Phenomenex Gemini C18 5μ 110 Å, 10×250 mm

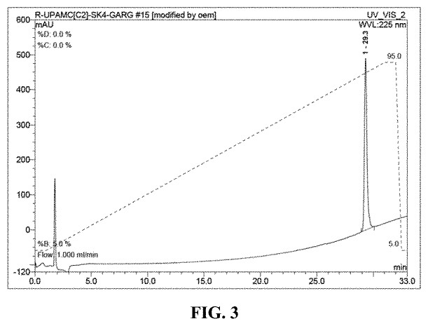

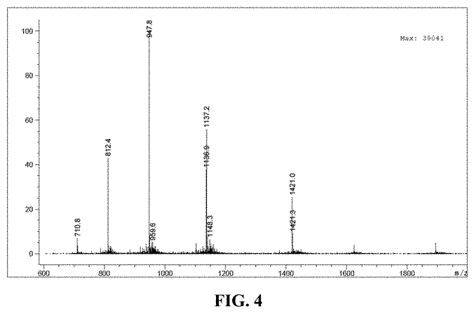

Eluent A: Water/0.1% TFA (v/v); Eluent B: MeCN/0.1% TFA (v/v)
Gradient: 0-1 min, 50% B; 1-2 min, 82% B; 2-10 min 95% B; 10-10.5 min 50% B
Flow: 4 mL/min
Collected main peak eluting at 7-8 minutes.
Analysis of the purified peptide is shown in FIGS. 3 and 4.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition comprising a compound of formula (1): 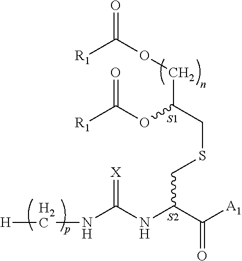
wherein:
each instance of R₁ is independently C5-21 aliphatic or C4-20 heteroaliphatic;
A₁ comprises an epitope;
X is O or S;
n is an integer selected from the group consisting of 2-5;
p is an integer selected from the group consisting of 0-15; and
S1 and S2 are each independently R or S;
or a pharmaceutically acceptable salt or solvate thereof.

2. The composition according to claim 1, wherein X is 0.

3. The composition according to claim 1 or claim 2, wherein p is 12.

4. The composition according to claim 1 or claim 2, wherein p is 14.

5. The composition according to any one of claims 1-4, wherein S1 is R.

6. The composition according to any one of claims 1-4, wherein S1 is S.

7. The composition according to any one of claims 1-6, wherein S2 is R.

8. The composition according to any one of claims 1-6, wherein S2 is S.

9. The composition according to claim 1, wherein: p is 14; n is 2; and S1 and S2 are R.

10. The composition according to claim 1, wherein: p is 14; n is 3; and S1 and S2 are R.

11. The composition according to claim 1, wherein: p is 14; n is 4; and S1 and S2 are R.

12. The composition according to claim 1, wherein: p is 14; n is 5; and S1 and S2 are R.

13. The composition according to claim 1, wherein: p is 14; n is 2; and S1 and S2 are S.

14. The composition according to claim 1, wherein A₁ comprises the amino acids of SEQ ID NO: 2 SKKKK.

15. The composition according to claim 1, wherein the composition further comprises at least one pharmaceutically acceptable carrier.

16. A method of provoking or enhancing an immune response in a subject, the method comprising administering to the subject an effective amount of the composition according to claim 1.
